(19) United States
(12) Patent Application Publication
(10) Pub. No.: US 2005/0288731 A1
(43) Pub. Date: Dec. 29, 2005

(54) Method and associated apparatus for feedback therapy

(76) Inventors: George H. Shames, Pittsburgh, PA (US); Carol A. Zimmerman, Macedonia, OH (US)

Correspondence Address: ECKERT SEAMANS CHERIN & MELLOTT, 600 GRANT STREET, 44TH FLOOR, PITTSBURGH, PA, 15219, US

(21) Appl. No.: 10/862,797

(22) Filed: Jun. 7, 2004

(51) Int. Cl. A61N 1/18

(52) U.S. Cl. 607/48

(57) ABSTRACT

A method of audio feedback and/or visual feedback therapy includes providing a microphone operatively associated with an audio delay unit, a plurality of trans-cutaneous nerve stimulation electrodes and a power supply. A patient speaks into the microphone thereby causing a responsive electrical signal representative of the patient's speech to pass through an audio delay which may be variable as to duration of delay and deliver the electrical feedback signals to the patient. Audio feedback and/or visual feedback may also be provided to the patient. Corresponding apparatus is disclosed.

### BACKGROUND OF THE INVENTION

1. Field of the Invention

The present invention relates to a method and associated apparatus for feedback therapy for treating patients for a wide variety of communication problems and more specifically it involves providing electrical feedback directly to the patient with or without audio and visual feedback.

2. Description of the Prior Art

Numerous means of treating patients having need for feedback therapy due to conditions such as stuttering, strokes, voice problems and numerous other causes have been known.

At the forefront of such treatment has been the use of those who by education and experience have specialized in counseling individuals frequently on a one-on-one basis in order to painstakingly and slowly bring about progress in the individual's desire to speak efficiently. Compounding the problems, such as stuttering, has been the awareness of the individual having the problem that frequent inability to speak creates awkward and embarrassing situations thereby making it desirable for the skilled counselor to treat not only the mechanical speech needs, but also the psychological needs of the patient.

Shames et al. U.S. Pat. No. 4,685,448 discloses a system for treating stutters wherein an individual speaking into a microphone has his or her speech amplified with tactile feedback being provided to the laryngeal or glottis region of the individual thereby providing a mechanical vibration in corresponding to the speech.

In spite of the foregoing known use of skilled therapists and equipment, there remains a very real and substantial need for improved methods and apparatus for treating people in need of feedback therapy which promotes smooth vocal cord vibration.

### SUMMARY OF THE INVENTION

The present invention has met the above-described needs.

In one embodiment, it employs the method wherein a microphone is operatively associated with an audio delay unit, a plurality of trans-cutaneous nerve stimulation electrodes and preferably with audio feedback and visual feedback units and an associated power supply. The patient emits sounds directed into the microphone which emits responsive electrical signals which are delayed by a predetermined amount which is adjustable and transmitted to provide an electrical signal to a plurality of trans-cutaneous nerve stimulation electrodes which is felt sub-cutaneously by the patient.

In addition to the electrical feedback, the patient may also receive audio feedback as through earphones and visual feedback by way of light located on an outer surface of the device that lights up and remains illuminated while the patient continuously vocalizes and shuts off when the patient interrupts or stops vocalizing. The objective is to allow the patient to receive tangible feedback regarding his or her continuous and sustained phonation.

It is preferred to provide two trans-cutaneous nerve stimulation electrodes, one of which is placed on the outer surface of the skin adjacent to the sternum and the other of which is placed on the outer surface of the skin approximately 0.5 to 1.5 inches from the first electrode (measured center-to-center). The electrical current flows from one electrode to the other thereby providing benign tickling feedback sensation as a result of his or her continuous phonation.

It is preferred to filter the electrical signal by a squelch system before it reaches the trans-cutaneous nerve stimulation electrode output or audio or visual output in order to minimize or eliminate undesired effects of ambient noise on the therapeutic feedback process.

Corresponding apparatus hereafter described is provided. The power supply preferably is operated by a battery. The apparatus is preferably miniaturized and disposed within a small housing so as to provide for portability.

It is an object for the present invention to provide a safe and effective method and associated apparatus for administering feedback therapy to people with a wide range of needs.

It is a further object of the present invention to employ patient-initiated sound production into corresponding electrical feedback from the trans-cutaneous nerve stimulation electrodes as a means for providing the patient with feedback regarding the sounds emitted by the patient. It is a further object of the present invention to combine electrical feedback with audio feedback and visual feedback.

It is a further object of the present invention to provide a miniaturized apparatus which is readily portable and energized by batteries.

It is yet another object of the invention to provide such a method and apparatus for a therapy which involves self-activated electrical, auditory and visual feedback as resulting from the emission by the speaker of sustained and continuous vocalization for communication problems.

It is yet another object of the present invention to provide such a system which is readily and reliably operable by the patient independently after initial consultation with a skilled practitioner.

These and other objects of the invention will be more fully understood from the following detailed description of the invention on reference to the illustrations appended hereto.

### BRIEF DESCRIPTION OF THE DRAWINGS

FIG. 1 is a schematic illustration of a preferred form of method of the present invention.

FIG. 2 is a schematic illustration of a preferred form of apparatus of the present invention.

### DESCRIPTION OF THE PREFERRED EMBODIMENTS

The method and apparatus of the present invention are designed to establish an awareness of vocal cord vibration on the part of the individual being treated. Vocal cord vibration is in turn converted into vocal sounds and finally converted into speech and language signals for purposes of communication and social interaction.

As employed herein, the term “feedback therapy” means a method and associated apparatus for treating a human being with the objective of improving speech including, but not limited to, treating individuals who stutter, have cerebral palsy, who have experienced a stroke, vocal abuse and other types of voicing problems such as pitch, volume and quality, people who are deaf or have hearing problems or other neuro-physiological problems or have a foreign dialect.

As employed herein, the term “patient” means a human being.

It will be appreciated that the present method and apparatus are adapted to treat a wide range of conditions which are somewhat diverse in nature. For example, stuttering can take many forms such as, for example, repetitions, pauses, blocking and locking the vocal cords together as a result of emotional stress. Stuttering tends to arise particularly during stress during the pre-school years, but also can occur when a child starts school or with the onset of puberty. By contrast, deafness is a very different type of physical condition which may have existed at birth or be the result of disease or even trauma.

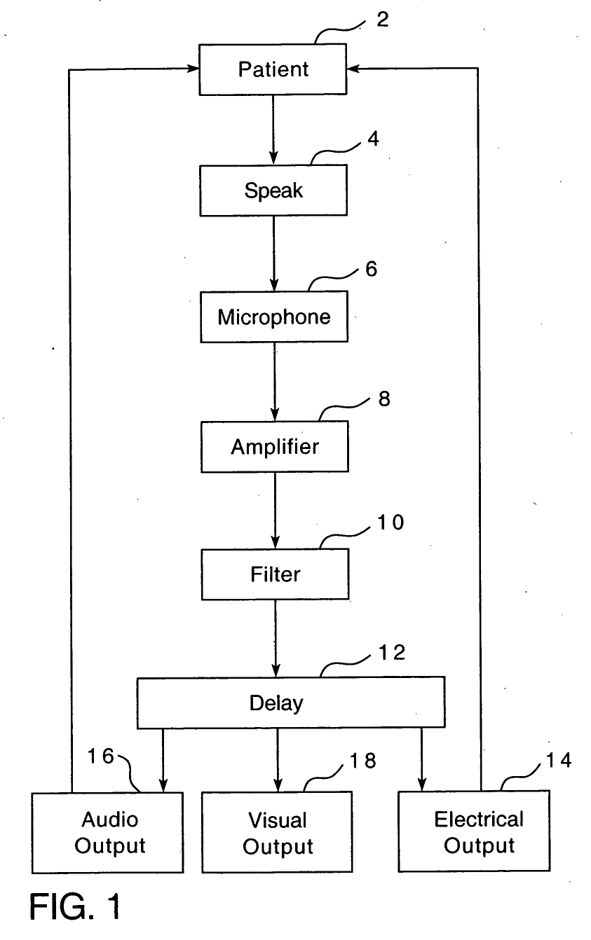

Referring to the preferred embodiment of the invention as set forth and shown schematically in the block diagram of FIG. 1, the patient 2 who is to receive feedback therapy speaks 4 into a microphone 6 with the emerging, corresponding electrical signal being amplified 8 and subsequently filtered 10 to eliminate annoying and interfering ambient noise signals which provide interfering signals that compete with the patient's vocal signals and erroneously activate the system. A squelch circuit may be employed to filter 10 for such undesired noise. An adjustable delay unit 12 which may, for example, have a range of delays from about 1 to 300 milliseconds receives the filter output. It will be appreciated that the delay may be provided on bucket brigade audio delay chips to provide the echo effect. The delay 12 is particularly helpful in treating conditions such as stuttering. It is a negative stimulus for slowing down and prolonging the patient's vocalizing thereby resisting or eliminating the echo effect. The output of the delay unit 12 is provided in the form of electrical output 14 to the patient by means of trans-cutaneous nerve stimulation electrodes which are a plurality of electrodes positioned at or adjacent the sternum and delivering an electrical signal. The electrical signal slows from one electrode to the other thereby providing the electrical feedback to the patient. In addition, particularly during the early stages of treatment, audio output 16 is also provided to the patient as by speakers delivering sound directly to the patient's ears, for example. In addition, visual output 18 may be provided to the patient by providing a light source 18 visible from the outside of the housing which housing is preferably miniaturized and made, for example, to fit into an individual's pocket or, if not, be of such size as to be readily portable. The light source is structured to go on so that a patient can see the light as he or she is phonating and goes off when he or she is not phonating.

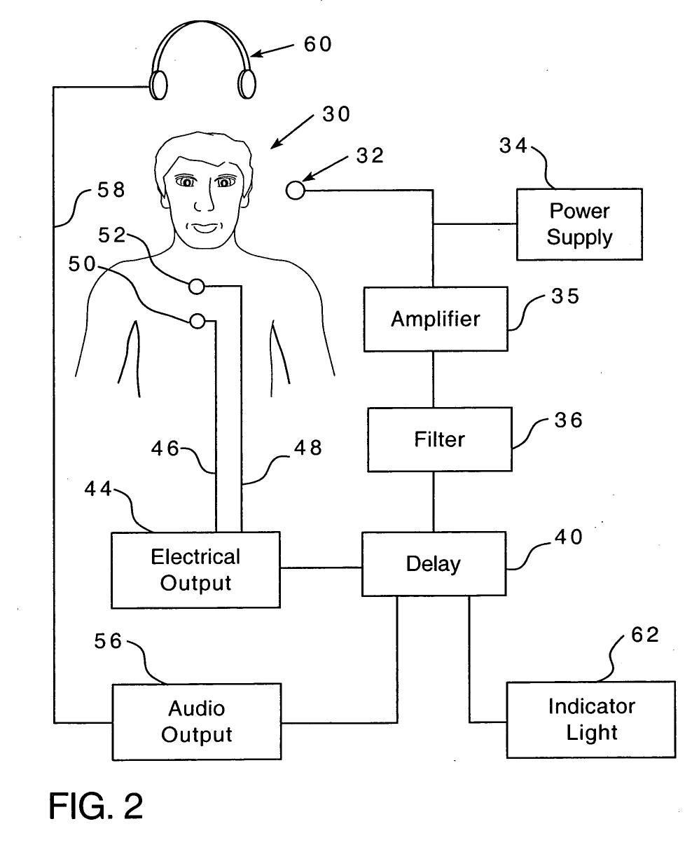

Referring to FIG. 2, there is shown schematically a patient 30 with a microphone 32 into which the patient speaks and an associated power supply 34, which may be a nine-volt battery, which serves to energize the electronics and power the trans-cutaneous nerve stimulation electrodes. The output electrical signal from the microphone 32 is amplified by amplifier 35, is filtered by filter 36 and that is subjected to the pre-programmed delay 40 after which the electrical output 44 is delivered by leads 46, 48 respectively to transcutaneous nerve stimulation electrodes 50, 52. One trans-cutaneous nerve stimulation electrode 50 is positioned adjacent the sternum of the patient with the other trans-cutaneous nerve stimulation electrode 52 being 0.5 to 1.5 inches therefrom measured between the electrode centers. In order to enhance the feedback, audio output is provided by unit 56 over lead 58 to headphones 60. After a period of treatment, the headphones 60 may be dispensed with the electrodes 50, 52 being the sole source of feedback. Visual feedback in the form of an indicator light 62 is provided in order to provide an indication that the individual is phonating when the light is “on” and is not phonating when it is “off”. After a period of treatment wherein the patient demonstrates his or her control over sustaining continuous sound production, the headphones 60 and indicator light 62 may be dispensed with and the electrodes 50, 52 become the sole source of feedback.

It will be appreciated that the present invention provides a safe and efficient means for providing feedback therapy to a patient by means of a miniaturized device which after proper patient counseling may be employed independently by the patient. The method and apparatus are structured to provide treatment for a wide range of patient communication problems.

Whereas particular embodiments of the invention have been described herein for purposes of illustration, it will be evident to those skilled in the art that numerous variations of the details may be made without departing from the invention as set forth in the appended claims.

1. A method of feedback therapy comprising
providing a microphone operatively associated with an audio delay unit,
a plurality of trans-cutaneous electronic nerve simulation electrodes and a power supply,
having a patient produce vocal sound into said microphone, and
delivering a signal corresponding to said vocal sound through an audio delay to said trans-cutaneous electronic nerve simulation electrodes to provide said patient with responsive electrical signals, whereby said patient will receive delayed electrical feedback representing said patient's speech.

2. The method of claim 1 including
providing said patient with feedback in addition to said trans-cutaneous electronic nerve stimulation electrodes which includes at least one feedback selected from the group consisting of audio feedback and visual feedback.

3. The method of claim 2 including
wherein said feedback is audio feedback.

4. The method of claim 2 including
wherein said feedback is visual feedback.

5. The method of claim 4 including
providing said visual feedback in the form of a light going on responsive to continuous phonation by the patient and said light being off when such phonation does not exist.

6. The method of claim 1 including
employing two said trans-cutaneous nerve simulation electrodes.

7. The method of claim 6 including
positioning a first said trans-cutaneous nerve simulation electrode on the patient generally at the patient's sternum.

8. The method of claim 7 including
providing a second said trans-cutaneous nerve simulation electrode adjacent said first trans-cutaneous nerve simulation electrode and spaced therefrom by about 0.5 to 1.5 inches measured between electrode centers.

9. The method of claim 8 including
securing said trans-cutaneous nerve simulation electrodes on the exterior of said patient's skin.

10. The method of claim 9 including
amplifying said signals before introducing said microphone output signal into said delay unit.

11. The method of claim 1 including
employing a battery in said power supply for energizing said apparatus.

12. The method of claim 10 including
filtering the output of said amplifier to reduce background noise.

13. The method of claim 1 including
simultaneously with said electrical feedback to said trans-cutaneous nerve stimulation electrode providing said patient with audio feedback.

14. The method of claim 6 including
employing said method to treat stuttering.

15. The method of claim 6 including
effecting said delay for about 1 to 300 milliseconds.

16. The method of claim 1 including
employing said method in phonation training.

17. Apparatus for feedback therapy comprising
a microphone for receiving vocal sound from a patient and emitting corresponding electrical signals,
a power supply for energizing said apparatus,
a plurality of trans-cutaneous nerve stimulation electrodes for receiving electrical signals corresponding to said speech and delivering electrical feedback to said patient, and
a delay unit for delaying delivery of said electrical signals to said transcutaneous nerve stimulation electrode.

18. The apparatus of claim 17 including
an audio output unit for delivering audio output to said patient responsive to said microphone output electrical signals.

19. The apparatus of claim 18 including
said delay unit being structured to effect said delay for about 1 to 300 milliseconds in delivering said electrical signals to said trans-cutaneous nerve stimulation electrodes.

20. The apparatus of claim 19 including
an indicator light for delivering visual output responsive to said microphone output electrical signal.

21. The apparatus of claim 19 including
said delay unit delaying delivery of said audio output by substantially the same delay as the delay employed in delivery of said electrical signals.

22. The apparatus of claim 19 including
said indicator light output being delayed substantially by the same delay as the delay employed in delivery of said electrical signals.

23. The apparatus of claim 19 including
a filter for reducing background noise from said microphone electric signals.

24. The apparatus of claim 17 including
said apparatus having two said trans-cutaneous nerve stimulation electrodes.

25. The apparatus of claim 17 including
said trans-cutaneous nerve stimulation electrodes being structured to be secured to said patient adjacent said patient's sternum.

26. The apparatus of claim 17 including
said trans-cutaneous nerve stimulation electrodes being structured to be secured to said patient approximately 0.5 to 1.5 inches from each other measured between the electrode centers.

27. The apparatus of claim 18 including
said audio output delivered to said patient through earphones.
